(19) United States
(12) Patent Application Publication
(10) Pub. No.: US 2002/0038121 A1
(43) Pub. Date: Mar. 28, 2002

(54) Apparatus system and for identifying a treatment tool within a patient's body

(76) Inventors: Ytzhak Rozenberg, Ramat Gan (IL); Shimon Eckhouse, Haifa (IL); Uzi Gil, Rishon Le Zion (IL)

Correspondence Address: Eitan, Pearl, Latzer & Cohen-Zedek, One Crystal Park, 2011 Crystal Drive, Suite 210, Arlington, VA, 22202-3709, US

(21) Appl. No.: 09/956,245

(22) Filed: Sep. 20, 2001

Related U.S. Application Data
(60) Provisional application No. 60/234,195, filed on Sep. 20, 2000.

(51) Int. Cl. A61B 18/18

(52) U.S. Cl. 606/15

(57) ABSTRACT

An apparatus, system and method for determining the location of a treatment tool within a body using a waveguide coupled to the treatment tool and adapted to carry electromagnetic radiation to a point of egress near a predefined point on the treatment tool. The waveguide being connected to a radiation source which may produce and transmit electromagnetic radiation along the waveguide.

### RELATED APPLICATIONS

This application claims priority from U.S. Provisional Patent Application Serial No. 60/234,195, filed on Sep. 20, 2000, which is hereby incorporated by reference in its entirety.

### FIELD OF THE INVENTION

The present invention relates generally to endoscopic and endovascular surgical tools. More particularly, the present invention relates to an apparatus system and method for identifying the location of a treatment tool within the body of a patient.

### BACKGROUND

In modern medicine it is common to insert a treatment tool into the body of a patient. Such medical tools and techniques are designed to be minimally invasive, target only an affected area and are usually less traumatizing than surgical techniques or procedures of the past. In many surgical techniques, e.g., endoscopic surgery, a small opening is made in the patient, and one or more catheters or cannulas are inserted into the opening. By using various working passageways small surgical instruments may be advanced into a location within the patient's body that is in close proximity or in contact with the target tissue.

It will be clear to those skilled in the art that numerous other, well known, medical procedures or techniques utilize treatment tools which are inserted into the body of a patient to specifically treat the affected tissue or the affected area. This group of medical procedures or techniques will be collectively referred to hereinafter as Micro Procedures.

One need, which arises when performing the Micro Procedures described above, is to identify the current location of the treatment tool within the patient's body. Determining the location of a treatment tool within a patient's body is desired for enabling the treating doctor to direct the treatment tool to the desired location—the treatment site. Today, determining the location of the treatment tool is commonly achieved by utilizing X-ray and/or Ultrasound procedures. X-ray and Ultrasound imaging techniques require special and expensive equipment, and in addition may also require the presence of another person, besides the treating doctor.

There is, therefore, a need to provide a simple and inexpensive apparatus for indicating the location of a Micro Procedure treatment tool inside a patient's body.

### SUMMARY OF THE INVENTION

As part of the present invention there may be a medical treatment tool including a electromagnetic waveguide coupled to the treatment tool. The optical waveguide may be adapted to emit light at a point of egress which is near a pre-selected location on the treatment tool. The waveguide may be connected to an electromagnetic radiation source which may produce and transmit electromagnetic radiation along the waveguide. Radiation emitted from a point of egress on the waveguide may be detected trough tissue, thereby indicating the location and/or the path of the treatment tool within a body.

### BRIEF DESCRIPTION OF THE DRAWINGS

The present invention will be understood and appreciated more fully from the following detailed description taken in conjunction with the appended drawings in which:

FIG. 1 is a schematic illustration with a cutaway view of a catheter having a waveguide according to the present invention;

FIG. 2 is a schematic illustration of a medical instrument used for venous ligation having a waveguide connected thereto according to the present invention.

FIG. 3 is a schematic illustration of an endoscopic laser with a waveguide according to the present invention.

FIG. 4 is a schematic illustration of an endovascular surgical tool with a waveguide according to the present invention inserted to a patient's body.

FIG. 5 is a catheter with a waveguide having multiple points of egress according to the present invention,

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

According to an embodiment of the present invention, the radiation source may be adapted to emit visible light in the near infrared range.

According to an embodiment of the present invention, the radiation source may be adapted to emit noncoherent light.

According to an embodiment of the present invention, the radiation source may be a diode laser, a diode block, or a light emitting diode (LED).

According to an embodiment of the present invention, the waveguide may be an optical fiber.

According to an embodiment of the present invention the treatment tool, may have an optical lens adapted to allow the treating doctor to view an area within a patient's body.

According to all embodiment of the present invention, the treatment tool may have a catheter having an electrode adapted to apply electrical current.

According to another embodiment of the present invention, the treatment tool may have a surgical laser device adapted to emit laser radiation.

According to yet another embodiment of the present invention, the treatment tool may have a mechanic cutting instrument adapted to cut tissue.

According to an embodiment of the present invention, the waveguide may be operatively connected to a control module.

According to an embodiment of the present invention, the waveguide may be operatively connected to a display unit adapted to display an area within a patient's body.

According to an embodiment of the present invention, the waveguide may be operatively connected to an electricity source.

According to an embodiment of the present invention, the waveguide is within the treatment tool and the treatment tool may have an aperture to allow the emission of radiation from the waveguide.

According to an embodiment of the present invention, the aperture may be fitted with an optical element such as a lens or a reflective surface.

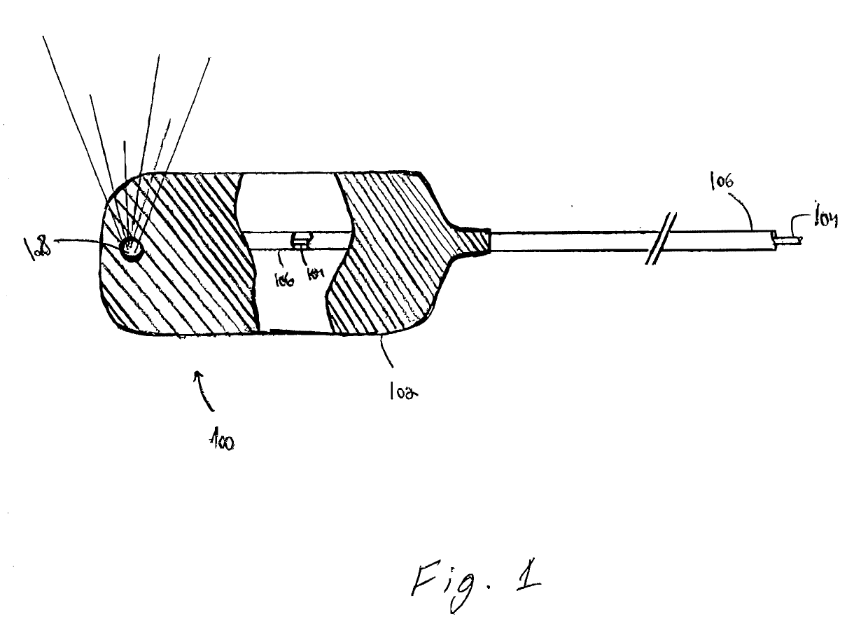

Turing to FIG. 1 there is shown a treatment tool 100 according to the present invention with a waveguide 104. The treatment tool 100 includes a catheter 102 and an optical waveguide 104 passing through both. The optical waveguide may be any one of a group of well known or yet to be devised optical pipes or treads adapted to conduct propagating electromagnetic radiation, for example an optical fiber. The optical waveguide may be donned within a sleeve 106. The sleeve 106 may provide insulation from heat or electric current or may facilitate the insertion and the propagation of the catheter 102 and the optical waveguide 104 inside a patient's body. The catheter 102 may have an aperture 108 at its distal end. The aperture at the distal end of the catheter 108 may coincide with a point of egress on the optical waveguide 104, such that light from the optical waveguide is emitted through the aperture 108.

The treatment tool 100 described hereinabove may be percutaneously advanced into a blood vessel or any other cavity, tube or pipe within the body of a patient, e.g., a varicose vein. Those of ordinary skill in the art may appreciate that the treatment tool may also be engaged according to some embodiments in conjunction with any endoscopic surgical or inspectiona tool known or yet to be devised. For example the treatment tool may be engaged with an electrode.

The optical waveguide 104 may be illuminating in red light, and may enable one to follow the position of the catheter in blood vessels which are approximately in the depth of 1-1.5 cm from the skin surface.

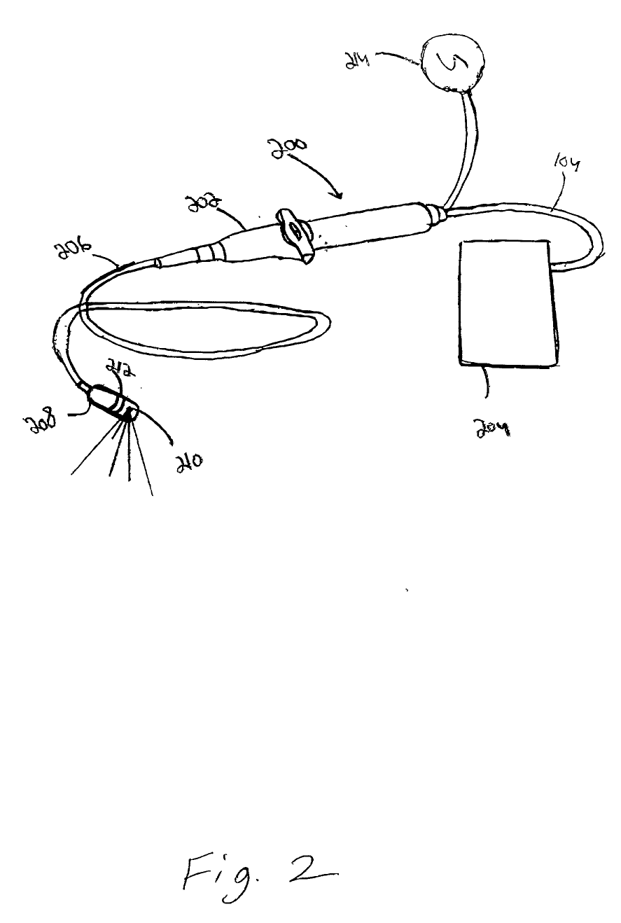

Referring to FIG. 2, a system 200 for an electric catheter 208 including an illumination fiber 104. The catheter 208 may be used for closure of blood vessels, for example in the venous ligation procedure described in U.S. Pat. No. 5,643,257. The catheter 208 further includes an electrode 212 for performing a treatment by passing electrical current to the blood vessel. The electric current may be provided by an electricity source 214. The electrode 212 can be both monopolar or bipolar. The catheter 208 can be for example of the type S.S. 302 of Unimade S.A, Switzerland.

The waveguide 104 may be connected to radiation source 204. Radiation produced by the radiation source may be transmitted along the waveguide, through a hand-piece 202, and may be emitted at a point of egrees along the waveguide. In FIG. 2, the point of egress is within the catheter 212. The waveguide's point of egress coincides with an aperture 210 in the catheter.

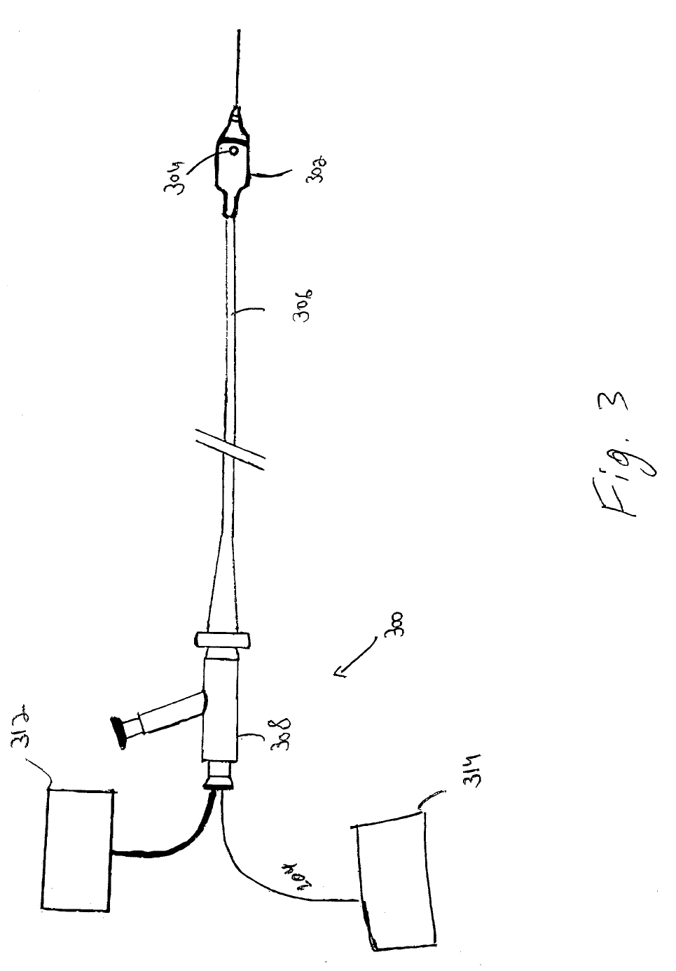

Referring to FIG. 3, there is shown a system for surgical operations 300, having an endoscope 308, a treatment laser source 312, an illuminating laser source 314, a sleeve 306 through which a waveguide 104 may be passed, and a treatment tip 302. Radiation from the treatment laser source 312 may be guided to the treatment 302 and may exit from the tip. Radiation from the illuminating radiation source 314 may pass though the waveguide 104 to the tip 304 and exit the tip via an egress point in the fiber and the aperture 304.

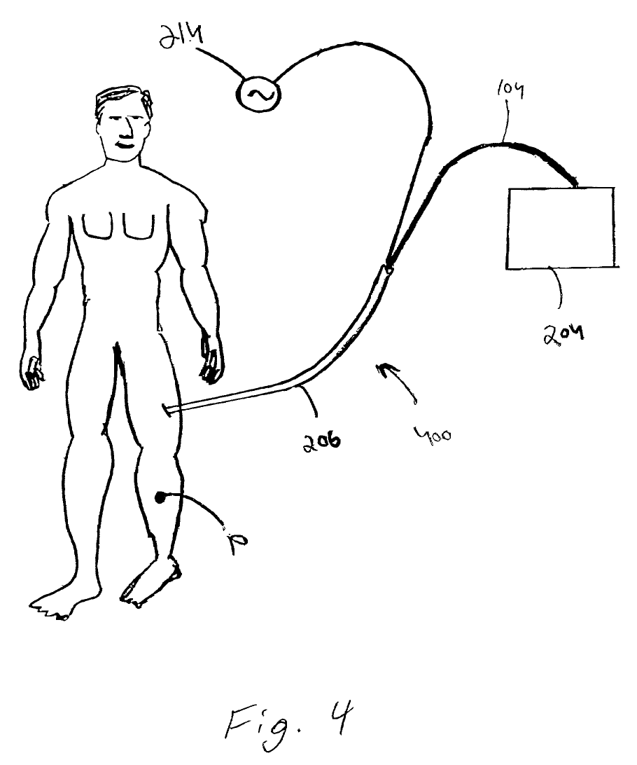

Referring to FIG. 4, there is shown the electric catheter of FIG. 2, being inserted into a body of a patient, Radiation produced by illuminating radiation source 204 passes though waveguide 104 and is emitted at a point of egress within the body of the patient. The point shown on the patent's lower leg and being designated as point P.

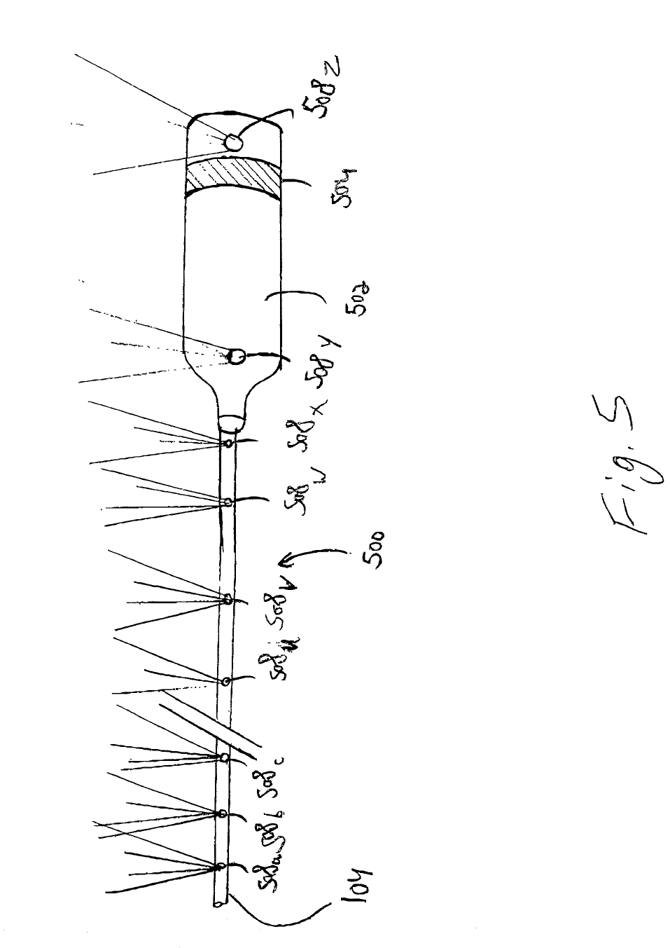

Referring to FIG. 5, there is shown a catheter 502 with a waveguide 104, where the waveguide 104 has multiple points of egress 508a through 508z.

It will be appreciated by persons skilled in the art that the present invention is not limited by what has been particularly shown and described herein above. Rather the scope of the invention is defined by the claims that follow:

What is claimed is:

1. An apparatus comprising a treatment tool and a waveguide coupled to said treatment tool such that the waveguide has a point of egress near a predefined point on said treatment apparatus.

2. The apparatus according to claim 1, wherein the waveguide is either an optical fiber or an optical tube.

3. The apparatus according to claim 2, wherein the waveguide has more than one egress points.

4. The apparatus according to claim 2, wherein the point of egress is near a distal end of said treatment tool.

5. The apparatus according to claim 2, wherein the treatment tool is selected from the group consisting of a catheter, a surgical laser, an electrode, an endoscope lens, a suction head, a mechanical tissue cutter, and a treatment agent applicator.

6. A system for determining the location of a treatment tool within a body comprising a waveguide operatively coupled to the treatment tool and adapted to carry electromagnetic radiation to a point of egress near a predefined point on the treatment tool, and a radiation source coupled to said waveguide and adapted to produce and transmit electromagnetic radiation along the waveguide.

7. The system according to claim 6, wherein the waveguide is selected from the group consisting of optical fiber and optical pipe.

8. The system according to claim 6, wherein the waveguide is adapted to carrier electromagnetic energy to more than one point of egress.

9. The system according to claim 6, wherein the predefined point on the treatment tool is near a distal end of the treatment tool.

10. The system according to claim 6, wherein said radiation source is selected from a group comprising a light emitting diode (LED), a diode laser, a diode laser block, a lamp, and a gas laser.

11. The system according to claim 6, wherein the radiation source is adapted to produce radiation in the visible near infrared range of the electromagnetic spectrum.

12. A method for determining the location of a treatment tool within a patient's body comprising transmitting along a waveguide electromagnetic energy to a point of egress near a predefined point on the treatment tool.

13. The method according to claim 12, further comprising observing the location of the electromagnetic radiation emitting from the point of egress.

14. The method according to claim 12, comprising transmitting electromagnetic radiation to more than one point of egress.

15. The method according to claim 12, wherein the electromagnetic radiation is within a visible near infrared range of the electromagnetic spectrum.
